SEQUENCE LISTING (4 sequences)

SEQ ID NO: 1 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 2 - DNA, 1434 bp, Homo sapiens
atggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctg

SEQ ID NO: 3 - protein, 459 aa, Homo sapiens
DQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 4 - DNA, 1377 bp, Homo sapiens
gaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctg

(19) United States
(12) Patent Application Publication
(10) Pub. No.: US 2015/0233921 A1
(43) Pub. Date: Aug. 20, 2015

(54) DETECTION OF SUBJECT BIOMARKER DIAGNOSTIC ASSAY FOR DENGUE FEVER AND THE DIFFERENTIATION OF DENGUE HEMORRHAGIC FEVER

(71) Applicants: Elizabeth Hunsperger, Guaynabo, PR (US); Momar Ndao, Montreal (CA); Kay Tomashek, San Juan, PR (US); B. Katherine Poole-Smith, San Juan, PR (US)

(72) Inventors: Elizabeth Hunsperger, Guaynabo, PR (US); Momar Ndao, Montreal (CA); Kay Tomashek, San Juan, PR (US); B. Katherine Poole-Smith, San Juan, PR (US)

(73) Assignee: The Government of the United States of America as represented by the Secretary of the Department of, Atlanta, GA (US)

(21) Appl. No.: 13/985,507

(22) PCT Filed: Feb. 16, 2012
(86) PCT No.: PCT/US12/25472

§ 371 Date: May 7, 2015

Related U.S. Application Data
(60) Provisional application No. 61/443,554, filed on Feb. 16, 2011.

(51) Int. Cl. G01N 33/569

(52) U.S. Cl. CPC G01N 33/56983; G01N 2333/4706

(57) ABSTRACT

Vitronectin is found to be a biomarker of progression from dengue fever to dengue hemorrhagic fever according to the present invention. Assay of vitronectin in biological samples of dengue virus infected subjects aids in prognosis, diagnosis and treatment of the disease, particularly in prediction of progression from dengue fever to severe dengue i.e. dengue hemorrhagic fever/dengue shock syndrome.

### REFERENCE TO RELATED APPLICATION

This application claims priority from U.S. Provisional Patent Application Ser. No. 61/443,554, filed Feb. 16, 2011, the entire content of which is incorporated herein by reference.

### GOVERNMENT INTEREST

The invention described herein may be manufactured, used, and licensed by or for the United States Government.

### FIELD OF THE INVENTION

The invention relates generally to disease diagnostics, and in particular to methods for ascertaining severity of dengue fever infection in a patient, differentiation of dengue fever and dengue hemorrhagic fever/dengue shock syndrome, and screening dengue fever therapeutics.

### BACKGROUND OF THE INVENTION

Dengue virus is a virus of the Flaviviridae family, genus Flavivirus. Four serotypes are known, DENY-1, DENV-2, DENV-3 and DENV-4.

The clinical course of dengue virus infection can be described as having several phases, shown schematically in FIG. 1. An incubation phase begins after a bite by a mosquito harboring the dengue virus and lasts from three to fourteen days, typically four to seven days. Following the incubation phase, viremia occurs and the dengue virus enters the bloodstream from the site of infection. During viremia, an acute febrile phase occurs, lasting two to seven days, typically three to five days. The acute febrile phase is followed by the critical phase, also called the afebrile phase, lasting one to three days, typically about two days. The patient will either recover or, occasionally, progress to dengue hemorrhagic fever/dengue shock syndrome.

Progression from dengue fever to dengue hemorrhagic fever/dengue shock syndrome is unpredictable. It is recognized that, following a first infection with dengue virus, infection with a second, third or fourth serotype of dengue virus is a risk factor for development of dengue hemorrhagic fever/dengue shock syndrome. Currently, there is a paucity of markers for progression from dengue fever to dengue hemorrhagic fever/dengue shock syndrome. Frequently, a patient with dengue fever will be sent home with instructions to return to the clinic or hospital if symptoms of dengue hemorrhagic fever occur, often with unfortunate consequences. In view of the severity and life-threatening nature of dengue hemorrhagic fever, a biomarker of progression from dengue fever to dengue hemorrhagic fever/dengue shock syndrome is required to aid in prognosis, diagnosis and treatment of the disease.

### SUMMARY OF THE INVENTION

Processes are provided according to aspects of the present invention for assessing dengue virus infection in a human subject including obtaining a biological sample from the human subject; and quantitating vitronectin in the sample, wherein the amount of vitronectin present in the sample is indicative of the severity of dengue virus infection in the human subject.

Processes are provided according to aspects of the present invention for assessing a febrile illness in a human subject including obtaining a serum, plasma or whole blood sample from the human subject; quantitating vitronectin in the sample to determine the level of vitronectin in the sample; and comparing the level of vitronectin with a standard or control to differentiate dengue fever or other febrile illness from severe dengue, i.e. dengue hemorrhagic fever/dengue shock syndrome.

Processes are provided according to aspects of the present invention for assessing dengue virus infection in a human subject including obtaining a biological sample from the human subject; and quantifying vitronectin by immunoassay and/or mass spectrometry.

Processes are provided according to aspects of the present invention for assessing dengue virus infection in a human subject including obtaining a biological sample from the human subject; and quantitating vitronectin in the sample, wherein the amount of vitronectin present in the sample is indicative of the severity of dengue virus infection in the human subject, wherein the biological sample is whole blood, plasma or serum.

Processes are provided according to aspects of the present invention for assessing dengue virus infection in a human subject including obtaining a biological sample from the human subject; purifying vitronectin from the biological sample to produce a purified sample; and quantitating vitronectin in the purified sample, wherein the amount of vitronectin present in the purified sample is indicative of the severity of dengue virus infection in the human subject, wherein the biological sample is whole blood, plasma or serum.

Processes are provided according to aspects of the present invention for assessing dengue virus infection in a human subject including obtaining a biological sample from the human subject; and quantifying vitronectin by ELISA or an antigen capture assay.

Processes are provided according to aspects of the present invention for assessing dengue virus infection in a human subject including obtaining a biological sample from the human subject; and quantifying vitronectin by lateral flow assay.

Processes for assessing dengue virus infection in a human subject are provided according to aspects of the present invention which include obtaining a first biological sample from the human subject at a first time during the acute febrile phase of dengue virus infection; obtaining a second biological sample from the human subject at a second time later than the first time during the acute febrile phase or critical phase of dengue virus infection; quantifying vitronectin in the first biological sample to obtain a first vitronectin level; quantifying vitronectin in the second biological sample to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Processes for assessing dengue virus infection in a human subject are provided according to aspects of the present invention which include obtaining a first biological sample from the human subject at a first time during the acute febrile phase of dengue virus infection; obtaining a second biological sample from the human subject at a second time later than the first time during the acute febrile phase or critical phase of dengue virus infection; quantifying vitronectin in the first biological sample by immunoassay and/or mass spectrometry to obtain a first vitronectin level; quantifying vitronectin in the second biological sample by immunoassay and/or mass spectrometry to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Processes for assessing dengue virus infection in a human subject are provided according to aspects of the present invention which include obtaining a first whole blood, plasma or serum sample from the human subject at a first time during the acute febrile phase of dengue virus infection; obtaining a second whole blood, plasma or serum sample from the human subject at a second time later than the first time during the acute febrile phase or critical phase of dengue virus infection; quantifying vitronectin in the first whole blood, plasma or serum sample to obtain a first vitronectin level; quantifying vitronectin in the second whole blood, plasma or serum sample to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Processes for assessing dengue virus infection in a human subject are provided according to aspects of the present invention which include obtaining a first whole blood, plasma or serum sample from the human subject at a first time during the acute febrile phase of dengue virus infection; obtaining a second whole blood, plasma or serum sample from the human subject at a second time later than the first time during the acute febrile phase or critical phase of dengue virus infection; purifying vitronectin from the first sample to obtain a first purified sample; purifying vitronectin from the second sample to obtain a second purified sample; quantifying vitronectin in the first purified sample to obtain a first vitronectin level; quantifying vitronectin in the second purified sample to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Processes for assessing dengue virus infection in a human subject are provided according to aspects of the present invention which include obtaining a first whole blood, plasma or serum sample from the human subject at a first time during the acute febrile phase of dengue virus infection; obtaining a second whole blood, plasma or serum sample from the human subject at a second time later than the first time during the acute febrile phase or critical phase of dengue virus infection; quantifying vitronectin in the first whole blood, plasma or serum sample by immunoassay and/or mass spectrometry to obtain a first vitronectin level; quantifying vitronectin in the second whole blood, plasma or serum sample by immunoassay and/or mass spectrometry to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Processes for assessing dengue virus infection in a human subject are provided according to aspects of the present invention which include obtaining a first whole blood, plasma or serum sample from the human subject at a first time during the acute febrile phase of dengue virus infection; obtaining a second whole blood, plasma or serum sample from the human subject at a second time later than the first time during the acute febrile phase or critical phase of dengue virus infection; quantifying vitronectin in the first whole blood, plasma or serum sample by ELBA or an antigen capture assay to obtain a first vitronectin level; quantifying vitronectin in the second whole blood, plasma or serum sample by ELISA or an antigen capture assay to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Processes for assessing dengue virus infection in a human subject are provided according to aspects of the present invention which include obtaining a first whole blood, plasma or serum sample from the human subject at a first time during the acute febrile phase of dengue virus infection; obtaining a second whole blood, plasma or serum sample from the human subject at a second time later than the first time during the acute febrile phase or critical phase of dengue virus infection; quantifying vitronectin in the first whole blood, plasma or serum sample by lateral flow assay to obtain a first vitronectin level; quantifying vitronectin in the second whole blood, plasma or serum sample by lateral flow assay to obtain a second vitronectin level; and comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

Vitronectin immunoassay devices are provided according to aspects of the present invention which include a solid or semi-solid porous support including a binding agent capable of specific binding to a first epitope of vitronectin.

Vitronectin immunoassay devices are provided according to aspects of the present invention which include a solid or semi-solid porous support including a binding agent capable of specific binding to a first epitope of vitronectin; and a conjugate pad comprising a detectably labeled binding agent capable of specific binding to a second epitope of vitronectin.

Vitronectin immunoassay devices are provided according to aspects of the present invention which include a solid or semi-solid porous support including a binding agent capable of specific binding to a first epitope of vitronectin; a conjugate pad comprising a detectably labeled binding agent capable of specific binding to a second epitope of vitronectin; and a wicking pad.

Vitronectin immunoassay devices are provided according to aspects of the present invention which include a solid or semi-solid porous support including a binding agent capable of specific binding to a first epitope of vitronectin; and a conjugate pad comprising a detectably labeled vitronectin.

Vitronectin immunoassay devices are provided according to aspects of the present invention which include a solid or semi-solid porous support including a binding agent capable of specific binding to a first epitope of vitronectin; a conjugate pad comprising a detectably labeled vitronectin; and a wicking pad.

Vitronectin immunoassay devices are provided according to aspects of the present invention which include a solid or semi-solid porous support including a binding agent capable of specific binding to a first epitope of vitronectin; and a housing at least partially enclosing the solid or semi-solid porous support.

Vitronectin immunoassay devices are provided according to aspects of the present invention which include a solid or semi-solid porous support including a binding agent capable of specific binding to a first epitope of vitronectin; a conjugate pad comprising a detectably labeled binding agent capable of specific binding to a second epitope of vitronectin; and a housing at least partially enclosing the the solid or semi-solid porous support and the conjugate pad.

Vitronectin immunoassay devices are provided according to aspects of the present invention which include a solid or semi-solid porous support including a binding agent capable of specific binding to a first epitope of vitronectin; a conjugate pad comprising a detectably labeled binding agent capable of specific binding to a second epitope of vitronectin; and a wicking pad.

Vitronectin immunoassay devices are provided according to aspects of the present invention which include a solid or semi-solid porous support including a binding agent capable of specific binding to a first epitope of vitronectin; a conjugate pad comprising a detectably labeled binding agent capable of specific binding to a second epitope of vitronectin; a wicking pad; and a housing at least partially enclosing the conjugate pad, the solid or semi-solid porous support, and the wicking pad.

### BRIEF DESCRIPTION OF THE DRAWINGS

FIG. 1 is a schematic illustration of phases of dengue virus infection;

FIG. 2A is a schematic illustration of a side view of a lateral flow device according to aspects of the present invention;

FIG. 2B is a schematic illustration of a side view of a lateral flow device according to aspects of the present invention;

FIG. 3 is a plot showing vitronectin levels in samples from dengue fever (DF (n=30)) and dengue hemorrhagic fever (DHF(n=30)) as detected by ELISA (*p<0.001) (DV−=Dengue virus negative samples);

FIG. 4 is an image of an immunoblot showing vitronectin isoform levels in patient samples;

FIG. 5 is a plot showing a comparison of vitronectin levels as detected by ELISA from Thailand study samples, where the legend includes dengue fever (DF), dengue hemorrhagic fever (DHF), and hepatitis C (HepC); 1° or 2° relate to clinical severity, while Gr defines a geographical and temporally infected clustered group of patients; and

FIG. 6 is a graph showing quantitated differences in vitronectin levels in biological samples obtained from dengue fever (DF) and dengue hemorrhagic fever (DHF) patients of different ages.

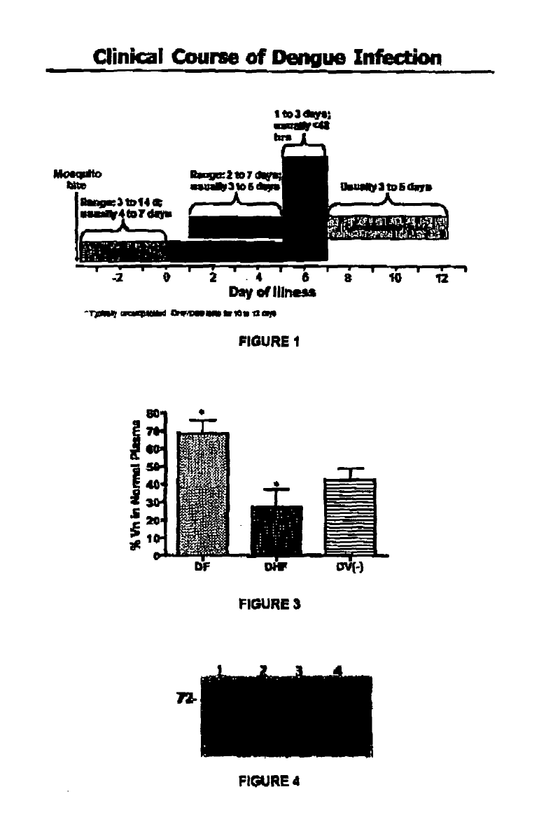

### DETAILED DESCRIPTION OF THE PREFERRED EMBODIMENTS

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Wild, D., The Immunoassay Handbook, 3rd Ed., Elsevier Science, 2005; Gosling, J. P., Imunoassays: A Practical Approach, Practical Approach Series, Oxford University Press, 2005; E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; Ormerod, M. G., Flow Cytometry: a practical approach, Oxford University Press, 2000; Givan, A. L., Flow Cytometry: first principles, Wiley, New York, 2001; and Herdewijn, P. (Ed.), Oligonucleotide Synthesis: Methods and Applications, Methods in Molecular Biology, Humana Press, 2004.

The singular terms “a,” “an,” and “the” are not intended to be limiting and include plural referents unless explicitly state or the context clearly indicates otherwise.

Assays for Assessment of Dengue Virus Infection

Processes for assessing dengue virus infection in a human subject are provided according to the present invention which include collecting a biological sample from the human subject; and quantifying vitronectin in the biological sample.

Vitronectin levels are increased in the biological sample of a human subject having dengue fever compared to a healthy human subject and vitronectin levels are decreased in a biological sample of a human subject having dengue hemorrhagic fever/dengue shock syndrome compared to a human subject having dengue fever or other febrile illness (OFI). Dengue hemorrhagic fever/dengue shock syndrome can be differentiated from dengue fever or other febrile illness by quantitation of vitronectin in a biological sample obtained from a human subject. According to aspects of the present invention, dengue hemorrhagic fever/dengue shock syndrome is differentiated from dengue fever or other febrile illness by quantitation of vitronectin in a serum, plasma or whole blood sample obtained from a human subject.

Vitronectin levels are increased in the biological sample of a human subject having secondary dengue fever compared to a healthy human subject and vitronectin levels are decreased in a biological sample of a human subject having secondary dengue hemorrhagic fever compared to a human subject having secondary dengue fever or other febrile illness. Secondary dengue hemorrhagic fever/dengue shock syndrome can be differentiated from dengue fever or other febrile illness by quantitation of vitronectin in a biological sample obtained from a human subject. According to aspects of the present invention, secondary dengue hemorrhagic fever/dengue shock syndrome is differentiated from dengue fever or other febrile illness by quantitation of vitronectin in a serum, plasma or whole blood sample obtained from a human subject.

Vitronectin levels are increased in the biological sample of a human subject age 15 years and older having dengue fever compared to a healthy human subject age 15 years and older and vitronectin levels are decreased in a biological sample of a human subject age 15 years and older having dengue hemorrhagic fever compared to a human subject age 15 years and older having dengue fever or a human subject age 15 years and older having another other febrile illness. Dengue hemorrhagic fever/dengue shock syndrome can be differentiated from dengue fever or other febrile illness by quantitation of vitronectin in a biological sample obtained from a human subject age 15 years and older. According to aspects of the present invention, dengue hemorrhagic fever/dengue shock syndrome is differentiated from dengue fever or other febrile illness by quantitation of vitronectin in a serum, plasma or whole blood sample obtained from a human subject age 15 years and older.

Vitronectin levels are increased in the biological sample of a human subject age 15 years and older having secondary dengue fever compared to a healthy human subject age 15 years and older and vitronectin levels are decreased in a biological sample of a human subject age 15 years and older having secondary dengue hemorrhagic fever compared to a human subject age 15 years and older having secondary dengue fever or a human subject age 15 years and older having another other febrile illness. Dengue hemorrhagic fever/dengue shock syndrome can be differentiated from secondary dengue fever or other febrile illness by quantitation of vitronectin in a biological sample obtained from a human subject age 15 years and older. According to aspects of the present invention, dengue hemorrhagic fever/dengue shock syndrome is differentiated from secondary dengue fever or other febrile illness by quantitation of vitronectin in a serum, plasma or whole blood sample obtained from a human subject age 15 years and older.

According to aspects of the invention, vitronectin is quantified in a sample obtained from a subject having a febrile illness. Where vitronectin in a sample obtained from the subject having a febrile illness is found to be decreased by 5%, 10%, 15%, 20%, 25%, 30%, 35%, 40%, 45%, 50%, 55%, 60%, 65%, 70%, 75%, 80%, 85%, 90%, 95% or more compared to a standard or level of vitronectin in a comparable sample obtained from a subject having dengue fever or other febrile illness, it is found that the subject's prognosis is poor and the disease is progressing from dengue fever to severe dengue fever, i.e. dengue hemorrhagic fever/dengue shock syndrome.

According to aspects of the invention, vitronectin is quantified in whole blood, plasma or serum samples obtained from a subject having a febrile illness. Where vitronectin in a whole blood, plasma or serum sample obtained from the subject having a febrile illness is found to be decreased by 5%, 10%, 15%, 20%, 25%, 30%, 35%, 40%, 45%, 50%, 55%, 60%, 65%, 70%, 75%, 80%, 85%, 90%, 95% or more compared to a standard or level of vitronectin in a whole blood, plasma or serum sample obtained from a subject having dengue fever or other febrile illness, it is found that the subject's prognosis is poor and the disease is progressing from dengue fever to severe dengue fever, i.e. dengue hemorrhagic fever/dengue shock syndrome.

According to aspects of the invention, vitronectin is quantified in whole blood, plasma or serum samples obtained from a subject having a febrile illness. Where vitronectin in a whole blood, plasma or serum sample obtained from the subject having a febrile illness is found to be decreased by 50% or more compared to a standard or level of vitronectin in a whole blood, plasma or serum sample obtained from a subject having dengue fever or other febrile illness, it is found that the subject's prognosis is poor and the disease is progressing from dengue fever to severe dengue fever, i.e. dengue hemorrhagic fever/dengue shock syndrome.

According to aspects of the invention, vitronectin is quantified in two or more samples obtained from a subject at different times. Where vitronectin in a sample obtained from a subject having dengue fever in the acute febrile or critical phase of dengue virus infection is found to be decreased by 5%, 10%, 15%, 20%, 25%, 30%, 35%, 40%, 45%, 50%, 55%, 60%, 65%, 70%, 75%, 80%, 85%, 90%, 95% or more compared to a level of vitronectin in a sample obtained from the subject at an earlier time in the clinical course of dengue fever in the patient, it is found that the subject's prognosis is poor and the disease is progressing from dengue fever to severe dengue fever, i.e. dengue hemorrhagic fever/dengue shock syndrome.

According to aspects of the invention, vitronectin is quantified in two or more whole blood, plasma or serum samples obtained from a subject at different times. Where vitronectin in a whole blood, plasma or serum sample obtained from a subject having dengue fever in the acute febrile or critical phase of dengue virus infection is found to be decreased by 5%, 10%, 15%, 20%, 25%, 30%, 35%, 40%, 45%, 50%, 55%, 60%, 65%, 70%, 75%, 80%, 85%, 90%, 95% or more compared to a level of vitronectin in a whole blood, plasma or serum sample obtained from the subject at an earlier time in the clinical course of dengue fever in the patient, it is found that the subject's prognosis is poor and the disease is progressing from dengue fever to severe dengue fever, i.e. dengue hemorrhagic fever/dengue shock syndrome.

A biological sample assayed for vitronectin according to processes of the invention may be any biological sample containing vitronectin including, whole blood, plasma, serum, extracellular fluid, cytosolic fluid, and tissue. According to aspects of the present invention, the biological sample is whole blood, plasma or serum.

The terms “subject” and “patient” are used interchangeably herein and refer to a human individual. It is appreciated that aspects of the present invention are applicable to non-human mammalian or avian subjects for dengue virus.

The terms “control” and “standard” are familiar to those of ordinary skill in the art and refer to any control or standard that can be used for comparison. The control or standard may be determined prior to assay for vitronectin, in parallel, simultaneously, in a multiplex assay or other assay format. A control or standard can be a first vitronectin level determined by assay in a first sample obtained from a patient. A control or standard can be a normal vitronectin level or range of vitronectin levels in a population of healthy subjects, dengue fever patients or severe dengue patients, for example.

Quantifying vitronectin in a biological sample according to aspects of the present invention is accomplished by assays including, but not limited to, a binding assay and/or mass spectrometry.

Binding assays include use of a binding agent to detect an anlayte.

The term “binding agent” as used herein refers to an agent characterized by substantially specific binding to a specified substance. The phrase “specific binding” and grammatical equivalents as used herein in reference to binding of a binding agent to a specified substance refers to binding of the binding agent to the specified substance without substantial binding to other substances present in a sample to be assayed for presence of the specified substance. It is understood by the ordinarily skilled artisan that specific binding refers to specific binding as determinable by use of appropriate controls to distinguish it from nonspecific binding.

Binding agents substantially specific for vitronectin may be obtained from commercial sources or generated for use in methods of the present invention according to well-known methodologies.

The term “binding” refers to a physical or chemical interaction between a binding agent and the target. Binding includes, but is not limited to, ionic bonding, non-ionic bonding, covalent bonding, hydrogen bonding, hydrophobic interaction, hydrophilic interaction, and Van der Waals interaction.

Quantifying vitronectin in a biological sample according to aspects of the present invention may include detection of a detectable label directly or indirectly attached to vitronectin. The term “detectable label” refers to any atom or moiety that can provide a detectable signal and which can be attached to a binding agent or analyte. Examples of such detectable labels include fluorescent moieties, chemiluminescent moieties, bioluminescent moieties, ligands, particles, magnetic particles, fluorescent particles, colloidal gold, enzymes, enzyme substrates, radioisotopes and chromophores.

Any appropriate method, including but not limited to spectroscopic, optical, photochemical, biochemical, enzymatic, electrical and/or immunochemical is used to detect a detectable label in an assay described herein.

Immunoassays and nucleic acid hybridization assays are binding assays used to detect vitronectin in a biological sample obtained from a patient according to embodiments of the present invention.

Immunoassays are well-known in the art and include, but are not limited to, enzyme-linked immunosorbent assay (ELISA), immunochromatography, antigen capture, flow cytometry, immunoblot, immunoprecipitation, immunodiffusion, immunocytochemistry, radioimmunoassay, and combinations of any of these. Immunoassays for both qualitative and quantitative assay of a sample are described in detail in standard references, illustratively including Wild, D., The Immunoassay Handbook, 3rd Ed., Elsevier Science, 2005; Gosling, J. P., Imunoassays: A Practical Approach, Practical Approach Series, Oxford University Press, 2005; E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; Ormerod, M. G., Flow Cytometry: a practical approach, Oxford University Press, 2000; and Givan, A. L., Flow Cytometry: first principles, Wiley, New York, 2001.

Immunoassay according to aspects of the present invention may include contacting a solid phase support, which may be a semi-solid support, including an anti-vitronectin antibody and a biological sample to detect binding of the anti-vitronectin antibody and vitronectin in the biological sample. The substrate can be in any of various forms or shapes, including planar, such as but not limited to membranes, silicon chips, glass plates and dipsticks; or three dimensional such as but not limited to particles, microtiter plates, microtiter wells, pins and fibers.

A solid support, which includes semi-solid support, for attachment of a binding agent, can be any of various materials such as glass; plastic, such as polypropylene, polystyrene, nylon; paper; silicon; nitrocellulose; or any other material to which a binding agent can be attached for use in an assay.

In particular aspects, a solid support to which a binding agent is attached is a particle.

Particles to which a binding agent is bound can be any solid or semi-solid particles to which a binding agent can be attached and which are stable and insoluble under assay conditions. The particles can be of any shape, size, composition, or physiochemical characteristics compatible with assay conditions. The particle characteristics can be chosen so that the particle can be separated from fluid, e.g., on a filter with a particular pore size or by some other physical property, e.g., a magnetic property.

Microparticles, such as microbeads, used can have a diameter of less than one millimeter, for example, a size ranging from about 0.1 to about 1,000 micrometers in diameter, inclusive, such as about 3-25 microns in diameter, inclusive, or about 5-10 microns in diameter, inclusive. Nanoparticles, such as nanobeads used can have a diameter from about 1 nanometer (nm) to about 100,000 nm in diameter, inclusive, for example, a size ranging from about 10-1,000 nm, inclusive, or for example, a size ranging from 200-500 nm, inclusive. In certain embodiments, particles used are beads, particularly microbeads and nanobeads.

Particles to which a binding agent is bound are illustratively organic or inorganic particles, such as glass or metal and can be particles of a synthetic or naturally occurring polymer, such as polystyrene, polycarbonate, silicon, nylon, cellulose, agarose, dextran, and polyacrylamide. Particles are latex beads according to aspects of the present invention.

Particles to which a binding agent is bound are optionally encoded and distinguishable from other particles based on a characteristic such as color, reflective index and/or an imprinted or otherwise optically detectable pattern. For example, the particles may be encoded using optical, chemical, physical, or electronic tags. Encoded particles can contain or be attached to, one or more fluorophores which are distinguishable, for instance, by excitation and/or emission wavelength, emission intensity, excited state lifetime or a combination of these or other optical characteristics. Optical bar codes can be used to encode particles.

According to aspects of the present invention, immunoassay includes assay of vitronectin in a biological sample by an immunochromatography technique. Broadly described, immunochromatography techniques include flowing a fluid test sample containing or suspected of containing an analyte of interest along a solid or semi-solid support including an anti-analyte antibody to detect specific binding of the antibody and analyte.

According to aspects of the present invention, quantitation of vitronectin in a biological sample obtained from a subject includes antigen capture, such as by lateral flow assay.

According to aspects of the present invention, quantitating vitronectin in a biological sample obtained from a subject is performed by a lateral flow assay. A lateral flow assay according to aspects of the present invention includes flowing a biological sample obtained from a patient along a solid or semi-solid support including an anti-vitronectin binding agent to detect specific binding of the anti-vitronectin antibody and vitronectin in the biological sample.

The biological sample obtained from the patient may be diluted or processed to purify vitronectin prior to analysis.

A lateral flow assay according to aspects of the present invention includes flowing a biological sample obtained from a patient along a solid or semi-solid support including an anti-vitronectin binding agent, such as an antibody, in the presence of a competitor to detect competition for binding of the anti-vitronectin binding agent, such as an antibody, with vitronectin in the biological sample.

According to aspects of the present invention, a lateral flow assay process for quantitating vitronectin includes providing: a conjugate pad where detectably labeled anti-vitronectin binding agent, such as an antibody, or detectably labeled vitronectin is diffusibly bound, the conjugate pad adjacent a solid or semi-solid porous support which allows for lateral flow of the fluid biological sample and which has at least one test detection zone including a non-diffusibly bound detection reagent and at least one control zone including a non-diffusibly bound control reagent, the solid or semi-solid porous support adjacent a wicking pad that promotes the capillary flow of the fluid biological sample along a flow path including the conjugate pad and the solid or semi-solid porous support.

A non-diffusibly bound detection reagent is an anti-vitronectin binding agent, such as an antibody. According to aspects of the present invention in which the conjugate pad contains a detectably labeled anti-vitronectin binding agent, the detection reagent is non-competitive with the detectably labeled anti-vitronectin binding agent.

A biological sample obtained from a subject is applied to the conjugate pad. The biological sample obtained from the subject may be diluted or processed to purify vitronectin prior to application to the conjugate pad.

According to aspects where a detectably labeled vitronectin binding agent is included in the conjugate pad, the detectable label is detected in the test zone to quantitate vitronectin in the sample and greater amounts of detected detectable label are indicative of greater amounts of vitronectin in the sample. According to aspects where a detectably labeled vitronectin is included in the conjugate pad, the detectable label is detected in the test zone to quantitate vitronectin in the sample and lower amounts of detected detectable label are indicative of greater amounts of vitronectin in the sample.

One or more standards may be used to associate an amount of detected detectable label with an amount of vitronectin in a sample.

The conjugate pad is typically blocked to inhibit non-specific binding. A non-limiting example of a blocking solution is 10 mM Borate, 3% BSA, 1%, PVP-40, 0.25% Triton x-100, pH 8.

Any reaction or diluent buffer compatible with the sample, reagents and reaction can be used, including but not limited to phosphate buffered saline, sodium phosphate buffer, potassium phosphate buffer, Tris-HCl buffer, Tricine buffer and other buffers described herein.

The conjugate pad is disposed adjacent to the solid or semi-solid porous support and the solid or semi-solid porous support is disposed adjacent to the wicking pad. Each component, the conjugate pad, the solid or semi-solid porous support and the wicking pad has a top surface in substantially the same plane as the top surface of each other component. The conjugate pad, the solid or semi-solid porous support and the wicking pad may be attached together so that they may be moved as one unit. Alternatively or additionally, the conjugate pad, the solid or semi-solid porous support and the wicking pad may all be attached to a structural support; such as a backing material for support and so that they may be moved as one unit.

According to aspects of the present invention, a lateral flow assay device is provided including 1) a conjugate pad where detectably labeled anti-vitronectin antibody or detectably labeled vitronectin is diffusibly bound, 2) a solid or semi-solid porous support which allows for lateral flow of the fluid biological sample and which has at least one test detection zone including a non-diffusibly bound detection reagent and at least one control zone including a non-diffusibly bound control reagent, and 3) a wicking pad that allows for the capillary flow of the fluid biological sample.

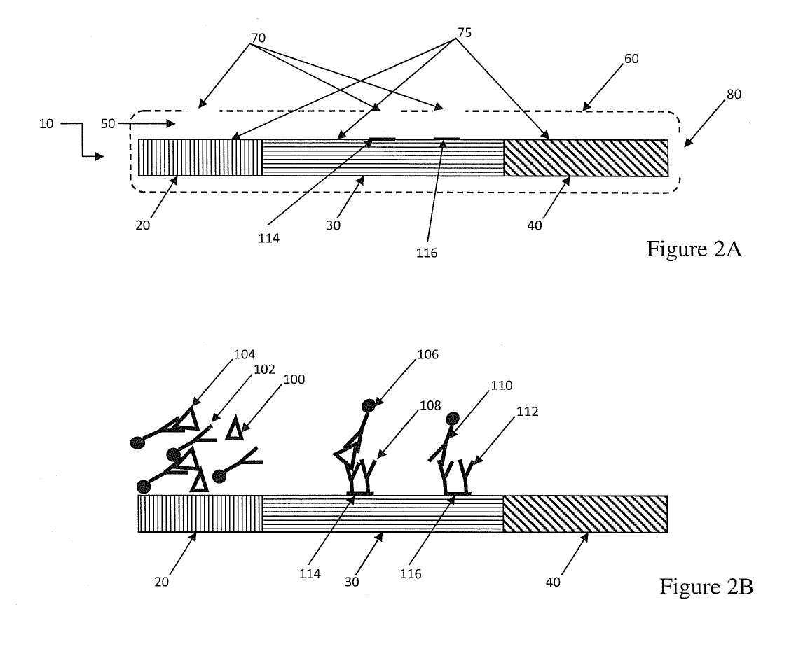

FIG. 2A is a schematic illustration of a device, 10, for lateral flow assay of vitronectin according to aspects of the present invention. The conjugate pad 20, solid or semi-solid porous support 30, and wicking pad 40, are attached and disposed adjacent to one another. The conjugate pad 20, solid or semi-solid porous support 30, and wicking pad 40 each have at least a top surface 75 substantially the same plane as each other top surface 75. The direction of lateral flow 50 is shown. An optional housing, 60, is shown which encloses the conjugate pad 20, solid or semi-solid porous support 30, and wicking pad 40. The housing optionally has one or more openings, 70, such as for application of a sample to be assayed for vitronectin or visualization of test and/or control results. The housing includes an opening 80 for insertion and removal of the conjugate pad 20, solid or semi-solid porous support 30, and wicking pad 40. A test zone 114 and a control zone 116 are shown.

FIG. 2B shows the conjugate pad 20, solid or semi-solid porous support 30, and wicking pad 40, test zone 114 and a control zone 116. A first detectably labeled binding agent capable of specific binding to vitronectin 102 is diffusibly attached to the conjugate pad 20. A test sample is added to the conjugate pad which contains or is suspected of containing vitronectin 100. The vitronectin and first detectably labeled binding agent capable of specific binding to vitronectin form a complex 104. The complex 104 is moved by lateral flow in the direction of the test zone 114 where it is bound to a second binding agent capable of specific binding to vitronectin which is non-competing with the first detectably labeled binding agent capable of specific binding to vitronectin, forming complex 106. Complex 106 is detected in the test zone by detection of the detectable label, thereby quantitating vitronectin in the test sample. Excess detectably labeled binding agent capable of specific binding to vitronectin 102 moves by lateral flow to the control zone where it binds to a binding agent 112 capable of specific binding to the binding agent capable of specific binding to vitronectin 102, forming a complex 110.

The term “diffusibly bound” refers to reversible attachment or adsorption of a material to the conjugate pad such that the material moves with the lateral flow when contacted with the biological sample. The term “non-diffusibly bound” refers to attachment of a material to the solid support wherein a non-diffusibly bound material is immobilized and therefore does not move with the lateral flow when contacted with the biological sample.

The term “test detection zone” refers to a region of the solid or semi-solid porous support where the detection reagent is non-diffusibly bound. The test detection zone may have any of various shapes and sizes configured to allow for determination of binding of an analyte to the detection reagent. Typically, the test detection zone is a line of non-diffusibly bound detection reagent, referred to as a “test line.”

The term “control zone” refers to a region of the solid or semi-solid porous support where the control reagent is non-diffusibly bound. The control zone may have any of various shapes and sizes configured to allow for determination of binding of a control substance to the control reagent. Typically, the control zone is a line of non-diffusibly bound control reagent, referred to as a “control line.”

A control reagent allows a user to confirm that the immunoassay is working properly. For example, a control reagent may be an antibody which specifically binds to the detectably labeled anti-vitronectin antibody.

According to aspects of the present invention, a lateral flow assay device includes 1) detectably labeled anti-vitronectin antibody diffusibly bound to the conjugate pad, 2) a solid or semi-solid porous support having a test detection zone including non-diffusibly bound anti-vitronectin antibody and 3) a wicking pad.

According to this aspect, the detectably labeled anti-vitronectin antibody diffusibly bound to the conjugate pad and the anti-vitronectin antibody non-diffusibly bound to the solid or semi-solid porous support bind specifically to different epitopes of vitronectin.

According to aspects of the present invention, a lateral flow assay device includes 1) a detectably labeled vitronectin epitope diffusibly bound to the conjugate pad, 2) a solid or semi-solid porous support having a test detection zone including non-diffusibly bound anti-vitronectin antibody and 3) a wicking pad. According to this aspect, the detectably labeled vitronectin epitope diffusibly bound to the conjugate pad binds specifically to the anti-vitronectin antibody non-diffusibly bound to the solid or semi-solid porous support and therefore competes with vitronectin in a test sample.

The conjugate pad is a material to which a detectably labeled vitronectin binding agent may be diffusibly attached including, but not limited to, glass fiber, bound glass fiber, polyester, cellulose and cellulose derivatives include cellulose acetate and nitrocellulose, nylon, polyvinylidene fluoride, polyethylene, polycarbonate, polypropylene, polyethersulfone and combinations of any of these.

The a solid or semi-solid porous support may be any solid or semi-solid adsorbent porous material suitable for chromatographic applications including, but not limited to, polyvinylidene fluoride, nylon, polyether sulfone, polyester, polypropylene, paper, silica, rayon, cellulose and cellulose derivatives include cellulose acetate and nitrocellulose, woven or non-woven natural or synthetic fibers and porous gels such as agarose, gelatin, dextran and silica gel. The solid or semi-solid porous support may be self-supporting, such as a membrane, or may be deposited on a structural support, such as an agarose thin layer deposited on a glass slide. According to aspects of the invention, the solid or semi-solid porous support is a nitrocellulose membrane.

The wicking pad is an absorbent material that facilitates lateral flow by wicking fluid including, but not limited to, an absorbent synthetic or natural polymer, such as cellulose.

A structural support to which the conjugate pad, solid or semi-solid porous support, and/or wicking pad are attached can be any material which provides support including, but not limited to, a backing card, glass, silica, ceramic and/or plastic membrane. An adhesive may be used to attach the conjugate pad, solid or semi-solid porous support, and/or wicking pad to the structural support.

A housing may be included to at least partially enclose the conjugate pad, solid or semi-solid porous support, and wicking pad. The housing may be configured to include a well for application of the fluid biological sample to the conjugate pad. The housing optionally allows the user to directly visualize assay results. Alternatively, the housing may include a detection device, such as an optical scanner, for detection of assay results.

The fluid biological sample flows by capillary action through the wicking pad to a control line and a test line that have binding agents, preferably antibodies, disposed at a precise concentration determined through validation experiments. The control line is an internal quality control that ensures the sample has migrated appropriately and validates the assay. The test line determines a positive or negative result for the analyte tested.

In particular aspects, an assay for vitronectin includes use of a mass spectrometry technique. For example, vitronectin can be ionized using an ionization method such as electrospray ionization (ESI), matrix-assisted laser desorption/ionization (MALDI) or surface enhanced laser desorption/ionization (SELDI). Mass analysis is conducted using, for example, time-of-flight (TOF) mass spectrometry or Fourier transform ion cyclotron resonance mass spectrometry. Mass spectrometry techniques are known in the art and exemplary detailed descriptions of methods for protein and/or peptide assay are found in Li J., et al., Clin Chem., 48(8):1296-304, 2002; Hortin, G. L., Clinical Chemistry 52: 1223-1237, 2006; Hortin, G. L., Clinical Chemistry 52: 1223-1237, 2006; A. L. Burlingame, et al. (Eds.), Mass Spectrometry in Biology and Medicine, Humana Press, 2000; and D. M. Desiderio, Mass Spectrometry of Peptides, CRC Press, 1990.

Vitronectin contained in a biological sample from a subject is optionally purified for assay according to a method of the present invention.

The term “purified” in the context of a biological sample refers to separation of a desired material in the biological sample from at least one other component present in the biological sample.

In particular embodiments, vitronectin is optionally substantially purified from the sample to produce a substantially purified sample for use in an inventive assay. The term “substantially purified” refers to a desired material separated from other substances naturally present in a sample obtained from the subject so that the desired material makes up at least about 1-100% of the mass, by weight, such as about 1%, 5%, 10%, 25%, 50% 75% or greater than about 75% of the mass, by weight, of the substantially purified sample.

Sample purification is achieved by techniques illustratively including electrophoretic methods such as gel electrophoresis and 2-D gel electrophoresis; chromatography methods such as HPLC, ion exchange chromatography, affinity chromatography, size exclusion chromatography, thin layer and paper chromatography. It is appreciated that electrophoresis and chromatographic methods can also be used to separate a peptide or peptides from other components in a sample in the course of performing an assay, as in, for example separation of proteins in immunoblot assays.

According to one aspect of the present invention, a subject biomarker is isolated and concentrated by absorption of vitronectin onto a solid substrate.

According to one aspect of the present invention, a subject biomarker is isolated and concentrated by binding to beads or other particles coupled with antibodies specific to vitronectin.

According to one aspect of the present invention, a subject biomarker is isolated and concentrated by binding to magnetic beads coupled with antibodies specific to vitronectin.

Compositions and methods are provided according to aspects of the present invention wherein a binding agent is an anti-vitronectin antibody. The term “antibody” is used herein in its broadest sense and includes single antibodies and mixtures of antibodies characterized by substantially specific binding to an antigen. An antibody provided according to compositions and methods is illustratively a polyclonal antibody, a monoclonal antibody, a chimeric antibody, a humanized antibody, and/or an antigen binding antibody fragment, for example. The term antibody refers to a standard intact immunoglobulin having four polypeptide chains including two heavy chains (H) and two light chains (L) linked by disulfide bonds in particular embodiments. Antigen binding antibody fragments illustratively include an Fab fragment, an Fab′ fragment, an F(ab′)2 fragment, an Fd fragment, an Fv fragment, an scFv fragment and a domain antibody (dAb), for example. In addition, the term antibody refers to antibodies of various classes including IgG, IgM, IgA, IgD and IgE, as well as subclasses, illustratively including for example human subclasses IgG1, IgG2, IgG3 and IgG4 and murine subclasses IgG1, IgG2, IgG2a. IgG2b, IgG3 and IgGM, for example.

In particular embodiments, an antibody which is characterized by substantially specific binding has a dissociation constant, Kd, less than about 10⁻⁷ M, such as less than about 10⁻⁸ M, less than about 10⁻⁹ M or less than about 10⁻¹⁰ M, or less depending on the specific composition. Binding affinity of an antibody can be determined by Scatchard analysis such as described in P. J. Munson and D. Rodbard, Anal. Biochem., 107:220-239, 1980 or by other methods such as Biomolecular Interaction Analysis using plasmon resonance.

Antibodies and methods for preparation of antibodies are well-known in the art.

Broadly, an immunogen is administered to an animal in particular methods, such as a rabbit, goat, mouse, rat, sheep or chicken and immunoglobulins produced in the animal are obtained from the animal, and optionally, purified for screening and use. An immunogenic fragment is a peptide or protein having about 4-500 amino acids, and in particular embodiments, at least 5 amino acids, or in further embodiments, at least 6, 7, 8, 9, 10, 11, 12, 13, 14, 15, 20, 22, 23, 24, 25, 30, 35, 40, 50, 100, 200, 300, or 400 amino acids.

Peptides and/or proteins used as immunogens may be conjugated to a carrier, such as keyhole limpet hemocyanin or bovine serum albumin.

Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

Monoclonal antibodies may be used in assays according to aspects of the present invention. Monoclonal antibodies are prepared using techniques known in the art such as described in E. Harlow and D. Lane, Antibodies: A Laboratory Manual, Cold Spring Harbor Laboratory Press, 1988; F. Breitling and S. Diibel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003, for example. Monoclonal antibodies according to the present invention and/or used in methods according to the present invention are produced by techniques illustratively including, but not limited to, hybridoma techniques, recombinant nucleic acid methodology and/or isolation from a phage library, for example as described in the above cited references. Monoclonal antibodies are advantageously used in particular embodiments due to the specificity of the binding of monoclonal antibodies which recognize a single epitope.

Particular methods of monoclonal antibody preparation include obtaining spleen cells from an animal immunized with an immunogen and fusing the antibody-secreting lymphocytes with myeloma or transformed cells to obtain a hybridoma cell capable of replicating indefinitely in culture.

Antibodies obtained are tested for substantially specific binding to the immunogen by methods illustratively including ELISA, Western blot and immunocytochemistry.

A binding agent can be a nucleic acid binding agent. A nucleic acid binding agent, such as, but not limited to, a nucleic acid probe or primer able to hybridize to a target vitronectin mRNA or cDNA can be used for detecting and/or quantifying vitronectin mRNA or cDNA encoding a vitronectin protein or a fragment thereof. A nucleic acid probe can be an oligonucleotide of at least 10, 15, 30, 50 or 100 nucleotides in length and sufficient to specifically hybridize under stringent conditions to vitronectin nucleic acid such as mRNA or cDNA or complementary sequence thereof. A nucleic acid primer can be an oligonucleotide of at least 10, 15 or 20 nucleotides in length and sufficient to specifically hybridize under stringent conditions to the mRNA or cDNA, or complementary sequence thereof.

According to aspects of the present invention, quantitating vitronectin in a biological sample obtained from a subject is performed by a nucleic acid assay technique including, but not limited to, Northern blot, Southern blot, RNase protection assay, dot blot and in situ hybridization. According to aspects of the present invention, quantitating vitronectin in a biological sample obtained from a subject is performed by a nucleic acid assay including a nucleic acid amplification technique such as, but not limited to, PCR, RT-PCR ligation-mediated PCR and phi-29 PCR. Nucleic acid assays are described in detail in standard references, illustratively including J. Sambrook and D. W. Russell, Molecular Cloning: A Laboratory Manual, Cold Spring Harbor Laboratory Press; 3rd Ed., 2001; F. M. Ausubel et al., Eds., Short Protocols in Molecular Biology, Current Protocols, Wiley, 2002; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

A binding agent can be an isolated non-immunoglobulin protein, peptide or nucleic acid which binds to a molecule of interest with substantial specificity. For example, a binding agent is illustratively an aptamer which substantially specifically binds to vitronectin. The term “aptamer” refers to a peptide and/or nucleic acid that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Similarly, peptide-based aptamers are characterized by specific binding to a target wherein the aptamer is not a naturally occurring ligand for the target. Techniques for identification and generation of peptide and nucleic acid aptamers is known in the art as described, for example, in F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

Diagnosis and Characterization of Dengue Virus Infection

According to aspects of processes of the present invention, a subject is diagnosed with dengue virus infection. Diagnosis of dengue virus infection is established by observation of signs and symptoms characteristic of dengue virus infection in combination with a patient history consistent with exposure to the mosquito vector for the virus; detection of dengue virus in a biological sample obtained from a subject suspected of being infected with dengue virus; and/or detection of antibodies to dengue virus in a biological sample obtained from a subject suspected of being infected with dengue virus. Dengue virus can be detected in a biological sample by isolation of the virus; nucleic acid hybridization methods including, but not limited to, Northern blot, Southern blot, RNase protection assay and dot blot; nucleic acid amplification methods, including, but not limited to, PCR, RT-PCR, ligation-mediated PCR and phi-29 PCR. Nucleic acid assays for both qualitative and quantitative assay of a nucleic acid in a sample are described in detail in standard references, illustratively including J. Sambrook and D. W. Russell, Molecular Cloning: A Laboratory Manual, Cold Spring Harbor Laboratory Press; 3rd Ed., 2001; F. M. Ausubel et al., Eds., Short Protocols in Molecular Biology, Current Protocols, Wiley, 2002; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004. Examples of assays to diagnose dengue virus infection are described in detail in Velathanthiria, V. et al., Dengue Bulletin, 30:191-196, 2006; Bai, Z. et al., J. Med. Microbiol., 57, 1547-1552, 2008; Johnson, B. W. et al., J. Clin. Microbiol., 43(10): 4977-4983, 2005; and Poloni et al., Virology J., 7:22, 2010.

Detection of antibodies to dengue virus in a biological sample obtained from a subject suspected of being infected with dengue virus includes serological methodologies including but not limited to ELISA, hemagglutination-inhibition, complement fixation, neutralization test, Plaque Reduction and Neutralization Test (PRNT), microneutralization PRNT, immunoglobulin M (IgM) antibody capture enzyme linked immunosorbent assay (MAC-ELISA), immunoglobulin G (IgG) enzyme linked immunosorbent assay (IgG ELISA) and NS1 ELISA based antigen assay, such as described in detail in Martin, D. A. et al., J. Clin. Microbiol., 38(5):1823-1826, 2000; Qiu L W, et al., Clin Vaccine Immunol., 16(1):88-95, 2009; Ding, X. et al., Clin Vaccine Immunol., 18(3):430-4, 2011; Wang, S. M., Am J Trop Med Hyg., 83(3): 690-695, 2010 Kittigul L, et al., Am. J. Trop. Med. Hyg., 59(3):352-6, 1998; and Clarke D. H., et al., Am. J. Trop. Med. Hyg., 7(5):561-73, 1958.

The term “primary dengue fever” refers to infection of a subject with a first serotype of dengue virus. The term “primary dengue hemorrhagic fever” refers to infection of a subject with a first serotype of dengue virus, which has progressed to the more severe manifestation of dengue fever, dengue hemorrhagic fever/dengue shock syndrome. The term “secondary dengue fever” refers to an infection of a subject with a subsequent dengue virus infection, particularly infection by a second, third or fourth serotype of dengue virus where the subject has previously been infected with first serotype of dengue virus. The term “secondary dengue hemorrhagic fever” refers to the more severe manifestation of secondary dengue fever, secondary dengue hemorrhagic fever/dengue shock syndrome, where the subject has previously been infected with a first serotype of dengue virus. As will be recognized by those of skill in the art, processes of the present invention applicable to secondary “secondary dengue fever” and “secondary dengue hemorrhagic fever” are also applicable to cases of infection of a subject with a third or fourth serotype of dengue virus since these subsequent infections are also known to increase risk of dengue hemorrhagic fever/dengue shock syndrome.

In order to determine whether dengue infection is a “primary” or “secondary” infection, immunoassay may be used to detect antibodies directed to dengue virus. Primary dengue infection is characterized by a slow and low titer antibody response. IgM antibody is the first immunoglobulin isotype to appear, usually around five days post-onset of symptoms. Anti-dengue IgG is detectable at low titer at the end of the first week of illness, and slowly increases. In contrast, during a secondary infection, antibody titers rise extremely rapidly and antibody reacts broadly with many flaviviruses. High levels of IgG are detectable even in the acute phase and they rise dramatically over the next two weeks. Primary versus secondary dengue infection can be determined using a simple algorithm. Samples negative for dengue virus IgG in the acute phase and positive for dengue virus IgG in the convalescent phase of the infection are primary dengue virus infections. Samples positive for dengue virus IgG in the acute phase and a 4 fold rise in dengue virus IgG titer in the convalescent phase (with at least a 7 day interval between the two samples) is a secondary dengue infection.

In particular, a hemagglutination inhibition test or ELISA is frequently used to detect anti-dengue virus antibodies in a subject. Where anti-dengue virus antibodies are detected, a new case of dengue fever or dengue hemorrhagic fever is determined to be secondary dengue fever or secondary dengue hemorrhagic fever.

Vitronectin

Vitronectin is produced as a 478 amino acid precursor protein including a 19 amino acid signal peptide. Mature vitronectin is a multifunctional glycoprotein with a full length sequence of 459 amino acids. The major source of vitronectin is the liver however it is present in blood and in the extracellular matrix. Vitronectin binds glycosaminoglycans, collagen, plasminogen and the urokinase-receptor, and also stabilizes the inhibitory conformation of plasminogen activation inhibitor-1 (PAI-1). It contains three glycosylation sites and exists in two major isoforms: a monomer (75 kDa) and a cleaved two-chain form (65 kDa+10 kDa) bound by a disulfide bond.

It is an aspect of the present invention that vitronectin has been observed in three different isoforms (75 kDa, 65 kDa and 54 kDa) in serum of dengue virus infected patients by the present inventors. Without wishing to be bound by theory, it is believed that the 54 kDa isoform is due to post-translational modifications including glycosylation and differences in cleavage. In addition, dengue virus infection may cause changes in post-translation modifications. Post-translational modifications include the addition of sulfate on 2 tyrosine residues and phosphorylation of a threonine 69 and 76. The size variation in vitronectin from 10 kDa (cleaved C-terminal domain) and 12 kDa (glycosylated 10 kDa C-terminal cleavage product) to 54 kDa (deglycosylated 75 kDa vitronectin monomer), 65 kDa (large cleavage product, glycosylated Stomatomedin B domain), and 75 kDa (uncleaved, fully glycosylated form of vitronectin) to even larger sizes (75 kDa+) also reflects protein complexes which function in vitronectin homeostasis such as: plasminogen activator inhibitor-1 (PAI-1), urokinase receptor, and insulin. VTN regulates blood coagulation by inhibiting the rapid inactivation of thrombin by antithrombin III in the presence of heparin detailed in Conlan, M. G. et al., (1988), Blood, 72(1): 185-190. In contrast to other adhesion proteins, vitronectin may participate in localized regulatory functions of blood coagulation as well as fibrinolysis in platelet-matrix interactions. Approximately 0.08% of the plasma derived vitronectin is found in platelets which may be released upon proper stimulation in different molecular forms, (i.e. as soluble protein and as a complex with PAI-1, as detailed in Preissner, K. T. et al., (1989), Blood, 74(6): 1989-1996.

In addition to the various isoforms, vitronectin can be found in complexes with other human proteins including: SERPINE 1 or serpin peptidase inhibitor (the N-terminal Stomatomedin B domain of Vnt interacts with PAI-1). Other complexes include: Stomatomedin B domain interaction with the urokinase receptor and vitronectin V75 interaction with heparin and insulin. These protein-vitronectin interactions are known as: vitronectin-PAI-1 complex, vitronectin-urokinase complex, vitronectin-heparin complex, and vitronectin-insulin complex.

Human vitronectin protein is also called V75, serum spreading factor, S-factor and Epiboin. Vitronectin consists of protein domains based on homology: one N-terminal Stomatomedin B, 4 hemopexin-like domains and a C-terminal domain with no known homology. When these domains are re-classified according to function, vitronectin also contains a heparin binding domain as detailed in Hayashi, M. et al., (1985). J. Biochem., 98(4): 1135-1138. Fragments of vitronectin are referred to as Stomatomedin B, hemopexin, or heparin-binding domain.

The cleavage of vitronectin into two-chain form (65 kDa+10 kDa) is mediated by a mutation to a positively charged residue, arginine at amino acid 379. This mutation is controlled by two alleles which are co-dominant in Caucasian populations as described in Akama, T., (1986), J. Biochem., 100(5): 1343-1351 and Conlan, M. G. et al., (1988), Blood, 72(1): 185-190. The result is three classes of vitronectin in Caucasians: 1-1, 1-2, and 2-2. The 1-1 class consists of uncleaved (75 kDa) vitronectin, the 1-2 class consists of both the cleaved (65-10 kDa) and uncleaved (75 kDa) vitronectin, and the 2-2 class is cleaved (65-10 kDa) vitronectin. The liver is the major source of plasma vitronectin suggesting that it may become depleted during disseminated intravascular coagulation (DIC), a symptom observed in many cases of dengue virus infected patients. Concentration of plasma vitronectin was remarkably reduced in some patients with DIC, especially in those with liver failure as described by Conlan, M. G. et al., (1988), Blood, 72(1): 185-190. Patients with vitronectin levels <40% normal had low fibrinogen and antithrombin III and a prolonged prothrombin expression. Plasma derived vitronectin polymorphism with ratios of the 75- and 65-kDa polypeptides isoforms of reduced vitronectin differed in disease versus normal controls as described in Conlan, M. G. et al., (1988), Blood, 72(1): 185-190. A significant decrease in plasma VTN levels is observed in chronic liver disease as described by Yamada, S., et al., (1999), Res. Commun. Mol. Pathol. Pharmacol., 104(3): 253-263 and the magnitude of the decrease seemed to correlate with the severity of the disease. Hepatic vitronectin levels increase in chronic liver disease, especially in the connective tissue around the portal and central veins and in the areas of piecemeal and focal necrosis.

Various isoforms of vitronectin may play a role in development of severe forms of dengue and dengue severity may vary between populations. According to the present invention, vitronectin is a biomarker for progression of dengue fever to severe dengue fever and the functional mechanism of vitronectin in severe dengue fever includes all of structural and functional domains and their various modifications through glycosylation, cleavage, and protein interactions as described.

The term “vitronectin” encompasses vitronectin precursor identified herein as SEQ ID NO:1, encoded by nucleic acid sequence SEQ ID NO:2; mature vitronectin identified herein as SEQ ID NO:3, encoded by nucleic acid sequence SEQ ID NO:4; as well as homologues and variants thereof.

Methods and compositions of the present invention are not limited to particular amino acid and nucleic sequences identified by SEQ ID NO herein and homologues and variants of a reference nucleic acid or protein may be used.

Homologues and variants of a nucleic acid or protein described herein are characterized by conserved functional properties compared to the corresponding nucleic acid or protein.

Vitronectin encompasses proteins having at least 80%, 85%, 90%, 91%, 92%, 93%, 94%, 95%, 96%, 97%, 98%, 99%, or 100% identity to the protein having the amino acid sequence set forth in SEQ ID NO:1, or a protein encoded by a nucleic acid sequence that hybridizes under high stringency hybridization conditions to the nucleic acid set forth in SEQ ID NO:2 or a complement thereof so long as the protein is characterized by functional properties of the protein of SEQ ID NO:1.

Vitronectin encompasses proteins having at least 80%, 85%, 90%, 91%, 92%, 93%, 94%, 95%, 96%, 97%, 98%, 99%, or 100% identity to the protein having the amino acid sequence set forth in SEQ ID NO:3, or a protein encoded by a nucleic acid sequence that hybridizes under high stringency hybridization conditions to the nucleic acid set forth in SEQ ID NO:4 or a complement thereof so long as the protein is characterized by functional properties of the protein of SEQ ID NO:3.

The term “vitronectin” refers to any fragment of a vitronectin that is operable in the described method utilizing the fragment, as understood by the ordinarily skilled artisan. A fragment of vitronectin is operative in any of the inventive methods described herein utilizing vitronectin.

“Vitronectin nucleic acid” as used herein refers to an isolated nucleic acid having a sequence that has at least 80%, 85%, 90%, 91%, 92%, 93%, 94%, 95%, 96%, 97%, 98%, or 99% identity to the nucleic acid sequence set forth in SEQ ID NO:2, or an isolated nucleic acid molecule having a sequence that hybridizes under high stringency hybridization conditions to the nucleic acid set forth in SEQ ID NO:2; or a complement thereof, so long as the nucleic acid effects the function described in the particular inventive method comprising use of the nucleic acid.

“Vitronectin nucleic acid” as used herein refers to an isolated nucleic acid having a sequence that has at least 80%, 85%, 90%, 91%, 92%, 93%, 94%, 95%, 96%, 97%, 98%, or 99% identity to the nucleic acid sequence set forth in SEQ ID NO:4, or an isolated nucleic acid molecule having a sequence that hybridizes under high stringency hybridization conditions to the nucleic acid set forth in SEQ ID NO:4; or a complement thereof, so long as the nucleic acid effects the function described in the particular inventive method comprising use of the nucleic acid.

A fragment of vitronectin nucleic acid is any fragment of a vitronectin DNA that is operable in the described method utilizing the fragment, as understood by the ordinarily skilled artisan. A fragment of vitronectin DNA is operative in any of the inventive methods described herein utilizing vitronectin nucleic acid.

The terms “complement” and “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

High stringency hybridization conditions are known to the ordinarily skilled artisan. An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone. Under highly stringent conditions, SEQ ID No. 2 will hybridize to the complement of substantially identical targets and not to unrelated sequences.

Percent identity is determined by comparison of amino acid or nucleic acid sequences, including a reference amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). The two sequences compared are generally the same length or nearly the same length.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264-2268, modified as in Karlin and Altschul, 1993, PNAS. 90:5873-5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, word length=12 to obtain nucleotide sequences homologous to a nucleic acid molecules of the present invention. BLAST protein searches are performed with the XBLAST program parameters set, e.g., to score 50, word length=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389-3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more nucleic acid or amino acid mutations can be introduced without altering the functional properties of a given nucleic acid or protein, respectively. Mutations can be introduced using standard molecular biology techniques, such as site-directed mutagenesis and PCR-mediated mutagenesis, to produce variants. For example, one or more amino acid substitutions, additions, or deletions can be made without altering the functional properties of a reference protein. Similarly, one or more nucleic acid substitutions, additions, or deletions can be made without altering the functional properties of a reference nucleic acid sequence.

When comparing a reference protein to a putative homologue, amino acid similarity may be considered in addition to identity of amino acids at corresponding positions in an amino acid sequence. “Amino acid similarity” refers to amino acid identity and conservative amino acid substitutions in a putative homologue compared to the corresponding amino acid positions in a reference protein.

Conservative amino acid substitutions can be made in reference proteins to produce variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size; alanine, cysteine, aspartate, glycine, asparagine, proline, threonine, serine, valine are all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

With regard to nucleic acids, it will be appreciated by those of skill in the art that due to the degenerate nature of the genetic code, multiple nucleic acid sequences can encode a particular protein, and that such alternate nucleic acids may be used in compositions and methods of the present invention.

Processes and substrates are provided to rapidly and reliably recognize infection by dengue virus in a human subject. The present invention provides methods for rapidly detecting the level of a subject biomarker of dengue virus infection in a subject.

Using a comparatively high concentration subject biomarker of DF or DHF viral infection allows for rapid detection and quantitation in a hospital or laboratory setting. The present invention has utility as a diagnostic test which guides patient treatment of dengue viral infection. The inventive test is rapid, highly sensitive, and distinguishes between DF and DHF. The instant invention also has utility as a tool for screening specific therapeutics compared to conventional dengue virus inhibitors and for monitoring infection response for vaccine candidate trials. The present invention has utility in monitoring dengue virus. The present invention affords a process to monitor onset, progression, and response to treatment kinetics of dengue virus infection, including the effectiveness of antiviral therapeutics.

Monitoring disease progression in a patient population is essential to providing optimal treatment following infective exposure to dengue virus. Often distinguishing an infection by dengue virus from other flu-like or febrile illnesses is difficult, particularly in a setting where exposure to dengue virus is rare. Early detection and identification of dengue virus is a benefit in tracking the infection mosquito population. Currently employed diagnostic techniques for identifying dengue virus infection are ineffective from 4 to 8 days post infection. The present inventive process employs several levels of specificity including specific immuno-adsorbance of the subject biomarker and substrates that are highly specific therefor. Thus, the present invention has the capability of detecting dengue virus infection less than 2 days post exposure. It is appreciated that the present invention offers results within 4 hours of obtaining a biological sample such that directed treatment strategies may begin earlier and enhancing potential patient survival.

The instant invention teaches a process for a subject biomarker assay for DF, DHF, and optionally differentiation therebetween in a biological sample. By using a subject biomarker for dengue virus infection, all serotypes of the virus are detected thereby overcoming a serious limitation of conventional serotype specific viral probes. The subject biomarker optionally is isolated and concentrated from the biological sample. The subject biomarker is subsequently reacted with a peptide substrate that is cleaved to yield at least two substrate cleavage products detected by one of several methods known in the art. As such, relative catalytic efficiency of the subject biomarker is measured.

The instant invention teaches a biological sample that is acquired by standard methods known in the art from a patient or other test subject illustratively including humans and other mammals. The biological sample illustratively includes whole blood, plasma, serum, extracellular fluid, cytosolic fluid, pleural fluid, or tissue.

A target form of subject biomarker is isolated and concentrated from the biological sample in an exemplary step through binding to beads coupled with an antibody specific to the subject biomarker. The beads employed are optionally magnetic, thereby allowing for gentle and rapid separation from other components present in the biological sample. The isolation and purification substrate occurs on a solid substrate or other substrates known in the art. A solid substrate is illustratively a microtiter plate. Magnetic beads are optionally coated with an antibody specific to the subject biomarker. Antibodies operative herein illustratively include those derived from organisms including mammal, mouse, rabbit, monkey, donkey, horse, rat, swine, cat, chicken, goat, guinea pig, hamster, and sheep. The antibody selected is appreciated to be monoclonal or polyclonal.

The instant invention teaches several detection methods, illustratively including mass spectrometry, fluorescence resonance energy transfer, fluorescence, light absorption, enzyme linked immunoadsorbant assay, coupled enzyme assay, continuous enzyme assay, discontinuous enzyme assay, flow cytometry, FLIPR, high-performance liquid chromatography, and colorimetric assay.

A biological sample is obtained from a patient or test subject and immediately sampled or alternatively frozen for later analysis at the situs of collection or remote from the source of the sample. A nonlimiting example includes samples taken in environments lacking state of the art diagnostic instruments. A simple blood sample is drawn into vacutainer or other tubes known in the art and then immediately frozen for prompt shipment. As a result, a diagnosis of infection is obtained in as little as 12-24 hours following a patient presenting symptoms of exposure to dengue virus.

A human subject biomarker operative herein for detection of DF or DHF and optionally differentiation therebetween includes vitronectin, plasminogen activator inhibitor-1 (PAI-1), tissue plasminogen activator, urokinase, and combinations thereof. It is appreciated that simultaneous detection of two or more human biomarkers is helpful in reducing false results. Preferably a human subject biomarker used in the present invention is vitronectin, alone or in combination with other subject biomarkers. As will be detailed hereafter, vitronectin levels in a human are statistically able to distinguish healthy, DF, and DHF.

An inventive kit employs prepackaged anti-subject biomarker coated beads to isolate the biomarker from a biological sample. A reaction chamber is provided for isolation and purification. Buffers are optionally included with the kit to be illustratively used for washing the beads, diluting the biological sample, eluting the beads, reacting with the peptide substrate, reconstituting the peptide substrate, storing the beads, storing the peptide substrate, freezing or otherwise storing the isolated and concentrated subject biomarker, freezing or otherwise storing the cleavage products, or preparing samples for detection. Suitable buffers illustratively include phosphate buffered saline (PBS), phosphate buffered saline plus Tween-20 (PBS-T), HEPES buffered saline (FIBS), HBS-Tween-20 (HBS-T), citrate-phosphate buffers, water, or other suitable buffers known in the art. The reaction chamber is used for cleavage of a peptide substrate. Optionally, a second reaction chamber is provided for cleavage of a peptide substrate. The isolated subject biomarker is appreciated to be amenable to freezing and shipment for remote analysis. It is further appreciated that cleavage products are also amenable to freezing for later detection, quantification, or analysis at a remote location and time. These or other methods of employing the present invention may be used to deliver rapid, effective diagnosis on a worldwide scale in a time frame that is not possible with current diagnostic techniques.

The inventive process is performed using numerous biological samples illustratively including whole blood, plasma, serum, extracellular fluid, cytosolic fluid, or tissue. Typically, serum is used as a suitable biological sample due to the ease in obtaining a sample by a venous blood draw from a patient. It is recognized in the art that numerous other biological samples are suitable in the present invention dependent on the application desired. By way of example, a biological sample may be as simple as an aqueous buffering agent such as FIBS or PBS, any of which are spiked with known or unknown levels of subject biomarker. Cell growth media is also suitable as a biological sample for screening transfected cell cultures for expression of active subject biomarker according to the present invention. It is appreciated that other biological samples are used such as a homogenized tissue sample that may or may not have been infected with dengue virus.

Upon selection of a biological sample, detecting subject biomarker by the present inventive process involves isolating and concentrating subject biomarker in the biological sample. Preferably, nonporous magnetic beads coated with antibodies that recognize and bind subject biomarker are employed to capture the subject biomarker from the biological sample. Magnetic beads have the advantage of requiring no centrifugation, thus allowing magnetic bead regeneration without loss of binding capacity. Magnetic beads also allow for minimal loss of sample due to pipetting as magnetic beads migrate to the sides of the reaction tube. It is further appreciated that magnetic beads allow for small scale isolation methods minimizing biological sample requirements. Other bead types or compositions operative herein illustratively include agarose, sepharose, nickel, or other materials known in the art. Numerous commercial sources are available for protein purification beads including New England Biolabs, Quiagen, and Bachem.

Coated magnetic beads suitable for use in the present inventive process are prepared and reacted with a suitable antibody for recognizing and binding subject biomarker. Monoclonal antibodies, polyclonal antibodies, or combinations thereof are suitable for selective subject biomarker binding. The antibodies are readily derived from numerous organisms including, but not limited to, a mouse, rabbit, monkey, donkey, horse, rat, swine, cat, chicken, goat, guinea pig, hamster, or sheep. Antibodies specific for subject biomarker are readily obtained from numerous commercial sources. The beads are then blocked with bovine serum albumin (BSA), polyethylene glycol (PEG), or other blocking agents known in the art. A biological sample is incubated with the anti-subject biomarker coated beads for sufficient time to allow equilibrium binding to develop, generally between 1 minute and 3 hours depending on the affinity of the antibody and the anticipated concentration of subject biomarker in the biological sample. Subject biomarker bound beads are then washed with a suitable buffer such as PBS-T, HBS-PEG, or other suitable buffering system known in the art to remove any unbound protein or other serum components. However, it is recognized in the art that the appropriate incubation time depends on substrate affinity, kinetic or catalytic efficiency constants intrinsic to the selected peptide substrate such that a detectable amount of product is formed in the incubation time. Such constants are readily determined by techniques well known and commonly practiced in the art.

Peptide substrates operative in the present inventive process are selected based on known affinity and kinetic constants as well as by the method of detection to be employed under the inventive method. Preferably, a peptide substrate possesses one potential scissile bond to simplify the kinetics of the cleavage reaction. The selected peptide substrate mimics the natural target of the subject biomarker or is a natural ligand of subject biomarker depending on the assay detection method to be employed. Typically the selected peptide is comprised of between 2 and 100 amino acid residues and preferably contains more than 10 residues. Preferably, the present invention is practiced with peptide substrates that mimic the sequence of the regions surrounding the scissile bond in natural subject biomarker target proteins. However, it is appreciated that other amino acid residues are optionally substituted within the sequence. For example, one or more amino acid residues within a sequence can be substituted by another amino acid of a similar polarity which acts as a functional equivalent. Substitutes for an amino acid within the sequence are illustratively selected from other members of the class to which the amino acid belongs. For example, the nonpolar (hydrophobic) amino acids include alanine, leucine, isoleucine, valine, praline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Also included within the scope of the present invention are ligands or fragments or derivatives thereof which are differentially modified during or after translation, e.g., by glycosylation, phosphorylation, acetylation, sulfation, linkage to an antibody molecule, or other cellular ligands.

It is also appreciated that an appropriate substitution is optionally employed that increases the interaction between the subject biomarker and a ligand or substrate therefor. A percent homology of greater than 50 percent is required and preferably greater than 90 percent. The percent homology is calculated by standard methods Current Methods in Sequence Comparison and Analysis,” Macromolecular Sequencing and Synthesis, Selected Methods and Applications, pp. 127-149, 1998, Alan R. Liss, Inc.

Alternatively, the peptide substrate for the subject biomarker is tagged with a biotin, avidin, horseradish peroxidase, streptavidin, or digoxin molecule. A nonlimiting example illustratively includes the addition of biotin to a residue within the peptide substrate such that upon cleavage a peptide of reduced size retains the biotin molecule(s) that is subsequently purified on an avidin column for further characterization or quantitation.

Alternatively, a substrate undergoes a colorimetric reaction. For example a substrate containing a p-nitroaniline or other group known in the art results in a color change in the solution following substrate cleavage by a subject biomarker. The creation of a species that modifies solution pH is also discernable through colorimetric monitoring of a pH indicator, or use of an ion selective electrode. Such a colorimetric assay can be performed either continuously or discontinuously and is further amenable to plate based assay formats similar to the FRET based or other fluorescence assays described above.

The present inventive method is amenable to numerous detection protocols and apparatus. In a preferred embodiment a sample of the analyte is analyzed by MALDI-TOF. MALDI-TOF has the advantage of recognizing particular cleavage products by resulting peptide masses. Comparison with an internal standard fixes the cleavage product mass. In a preferred embodiment an internal standard is an isotopically labeled peptide seven mass units higher than and corresponding to the sequence of the target cleavage peptide. Using a ratio of the area under the peak representing the target peptide and that representing the internal standard a relative quantity of the target peptide is obtained. Analyses of samples at numerous time points following addition of the peptide substrate to the reaction chamber allows for kinetic measurements of product formation and determination of the amount of subject biomarker present in the original biological sample. It is recognized in the art that numerous other forms of mass spectrometry may be employed as detection methods in the present invention such as electro-spray ionization LC/MS/MS, etc.

In another preferred embodiment detection of cleavage products is performed using a simple bench-top fluorometer. Employing dual labeled peptide substrate with a fluorescent group placed either N- or C-terminal to the scissile bond and a quenching group placed an appropriate distance from the fluorescent group on the opposite end of the scissile bond allows for rapid and real-time monitoring of reaction product formation following cleavage of the substrate reducing the FRET and resulting in an increase in observable fluorescence. Optionally, the reaction is quenched by the addition of 1 mM ortho-phenanthroline/10 mM EDTA after a known amount of time has elapsed following substrate addition to the reaction chamber. The magnitude of the fluorescence is measured and compared to a standard curve for determination of product formation per unit time that is then related back to the unknown activity of subject biomarker in the reaction. The endpoint analysis is particularly amenable to being performed in 96-well plate format for robotic processing and improving screening throughput. It is recognized in the art that both continuous and endpoint assay and detection methods are amenable to miniaturization to 384 well, 1096 well, or other plate based assay formats.

The present invention is also employed in screening protocols for the identification and trials of candidate vaccines by allowing rapid observation of the degree to which antibodies generated by a vaccine neutralize the effects of dengue virus infection on subject biomarker changes.

As the present invention capitalizes on the quantity of subject biomarker in a biological sample, it is operative to predict the disease progression in humans that have been subjected to dengue virus infection that may or may not have been pretreated with a vaccine candidate. A correlation is expected between the efficacy of a vaccine and levels of subject biomarker present in a biological sample from a test subject. As such, sampling subject tissues or fluid samples following the initiation of infection provides a real-time readout of the progress of the infection.

The following examples are not intended to limit the scope of the claimed invention and instead provide specific working embodiments. While the data provided is for vitronectin (Vn), it is appreciated that other subject biomarkers are readily analyzed in a like manner.

### EXAMPLES

### Example 1

### ELISA Analysis

The levels of endogenous Vn in serum are determined by ELISA assay using polyclonal Vn antibody as capture and mouse monoclonal as detection antibody. Color development is accomplished using anti-mouse horseradish peroxidase (HRP) conjugated Abs followed by tetramethyl benzidine (TMB) substrate incubation. Vn levels are calculated from a calibration curve based on vitronectin standards.

### Example 2

### Preparation of Beads

Antibody coated beads are obtained from a commercial source. 20-100 μl of bead suspension are used to covalently link anti-Vn Abs from a 100 μl sample to the beads according to the manufacturer's protocol. To separate the beads the reaction tube is placed on a magnet for 1 min and the resulting supernatant discarded by aspiration. The beads are resuspended in phosphate buffered saline with 0.05% Tween20, pH 7.3 (PBS-TW) and stored until ready for use. Thorough washing is achieved by repeating the magnetic pelleting and resuspension steps three times.

### Example 3

### Coating Beads with Desired Anti-Vitronectin Antibody

Anti-vitronectin coated magnetic beads (Vn-MABs) are prepared using mouse monoclonal anti-Vn IgG that is prepared according to the manufacturer's protocol using 40 ug IgG/100 ul magnetic bead suspension.

### Example 4

### Purification and Concentration of Vn from Serum

A serum, plasma, pleural fluid, or other biological sample is obtained from a patient. The sample is diluted in 500 μl PBS-TW and mixed gently with 20 μl Vn-MABs for 1 hour. The beads with Vn bound are retrieved, washed three times in PBS-TW, then reconstituted in PBS-TW for further analyses by mass spectrometry.

### Example 5

### Sample Testing

Samples are collected in Puerto Rico from 30 DHF adult patients, 30 DF adult patients, and dengue virus negative samples from adult patients presenting with a febrile illness (DV−). The standard used in the assay is a pool of more than 100 normal plasma donors and is assigned a value of 100%. The results for vitronectin levels detected by ELISA for the groups are provided in the plot of FIG. 3. In western blots, the DHF samples are noted to be almost devoid of the mature 75 kilodalton isoform of vitronectin in comparison with DF samples. The DHF samples were noted to include high levels of the 55 kilodalton precursor form of vitronectin.

FIG. 4 shows the result of analysis of vitronectin isoforms in pooled Puerto Rico (PR) samples of 2° dengue virus infections by Western blot analysis. Controls consisted of dengue virus (−) sample from PR and one OFI sample from Thailand. Lane 1, pool of dengue virus (−) PR samples; lane 2, pool of dengue fever (DF) PR samples; lane 3, pool of dengue hemorrhagic fever (DHF) PR samples; lane 4, OFI Thai sample. Samples from the more severe form of the disease, DHF, express more of the 54 kDa isoform when compared to DF samples and express less of the 75 kDa isoform compared to both PR DF and Thai OFI samples.

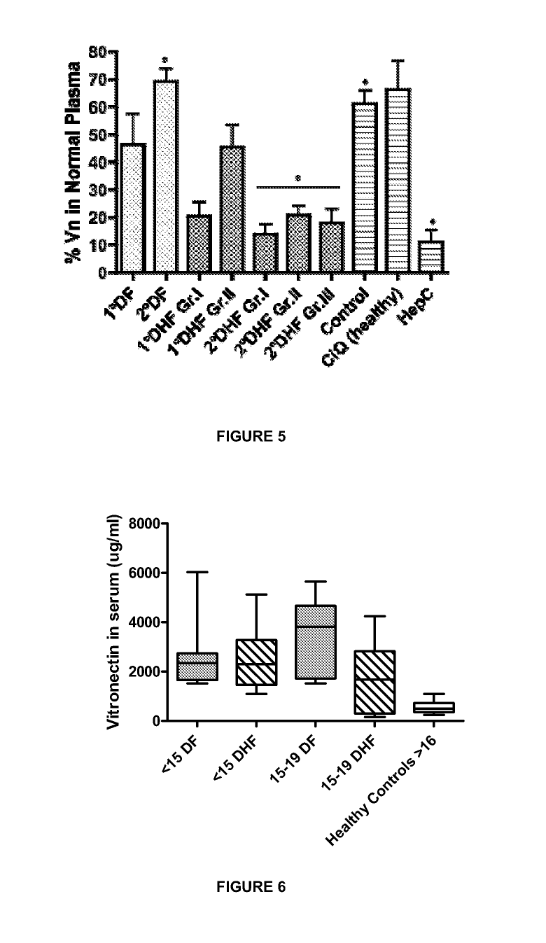

ELISA analysis was performed with serum samples for patients having varied degrees of DF or DHF with the samples coming from Thailand. As shown in FIG. 5, DHF patients express and display significantly lower levels of plasma vitronectin compared to DF patients. The vitronectin levels of DF patients in the Thailand sample group agree with the Puerto Rican study in that DF patients' vitronectin levels exceed those of healthy subjects. In FIG. 5, in addition to plotting CIQ (healthy) subjects which are equivalent to dengue virus- in FIG. 3, patients suffering from hepatitis C vitronectin levels are also plotted as a positive control.

### Example 6

### Lateral Flow Assay

According to embodiments of the present invention, vitronectin is a biomarker used as a prognostic diagnostic for dengue virus infection severity. A lateral flow assay is used according to embodiments to determine the levels of vitronectin. The lateral flow assay was optimized and developed based on the buffer used for the flow, the conjugated antibodies and the capture antibodies for vitronectin.

The detectable label used in this example is a 40 nm Gold conjugate, OD10. This detectable label is attached to an anti-vitronectin IgM antibody obtained commercially from Sigma, V7881. The anti-vitronectin IgM antibody is used at a concentration of 12 ug/ml. This detectable label is also attached to an anti-vitronectin IgG antibody obtained commercially from Innovative Research, IHVN1H820. The anti-vitronectin IgG antibody is used at a concentration of 10 ug/ml (IgG) anti-VTN, Innovative Research, IHVN1H820

Strips of various solid or semi-solid porous supports are tested for use in lateral flow assays to quantitate vitronectin. The strips are 60 mm×5 mm pieces of nitrocellulose membrane: HF180, Millipore, SHF1800425; HF090, Millipore, SHF0900225; CN140, Sartorius, 1UN14AR050025; or AE99, Whatman 10548081

The test detection zone is a test line applied to the nitrocellulose membrane as a single pass application of 0.7 mg/ml in 10 mM Sodium Phosphate, pH 7.7, or as a double pass application resulting in deposition of 1.4 mg/ml IgG. A microliter spot of 0.5 mg/ml IgM in 10 mM Sodium Phosphate, pH 7.7 is applied as a negative control.

The control detection zone is a control line of 0.5 mg/ml Goat anti-Mouse(GAM) IgG in 1XPBS, Quad Five, 4010101 applied to the membrane.

The nitrocellulose membrane is blocked with 10 mM Sodium Phosphate, 0.1% Sucrose, 0.1% Bovine Serum Albumin (BSA), 0.25% Polyvinylpyrrolidone (MW 40,000) (PVP-40), pH 7.2

The conjugate pad is a glass fiber conjugate pad, available commercially as G041, Millipore, or GF33, Whatman, a bound glass fiber conjugate pad available commercially as Standard 17, Whatman, or a polyester conjugate pad available commercially as Ahlstrom 6615, Ahlstrom.

The conjugate pad is blocked with 10 mM Borate, 3% BSA, 1%, PVP-40, 0.25% Triton x-100, pH 8.

A cellulose fiber wicking pad used is available commercially as C083, Millipore.

A structural support used is backing card available commercially as MIBA-020.

Several sample diluents are tested to optimize the lateral flow assay including 1) “PBS+” which is 1× Phosphate Buffered Saline (PBS), 0.01% Tween-20, 0.1% BSA, 0.01% sodium azide; HIV Running Buffer which is 25 mM Tris, 1% pentasodium tripolyphosphate, 0.1% sodium azide, 0.1% Triton X-405, 2 mM ethylenediaminetetraacetic acid (EDTA), 0.5% casein, pH 8; Running Buffer A which is 10 mM sodium phosphate, 0.1% sucrose, 0.1% fish gel, 0.25% PVP-40; Running Buffer B which is 200 mM borate, 150 mM sodium chloride (NaCl), 1% casein, 0.1% Tween-20, pH 8.3; and Running Buffer C which is 1× PBS, 0.1% Tween-20.

The target analyte is vitronectin and patient serum samples are used and compared to a commercially obtained standard virtonectin, Sigma, V8379.

Dipstick testing with wet gold conjugate is used to assay vitronectin in patient serum samples in this example. Culture tubes (VWR, 60818-306) are arranged vertically in a test tube rack. In each tube, 150 microliters of the positive or negative control is added. For testing serum, 10 microliters of serum is mixed with 140 microliters of buffer. The test tubes may be swirled gently or a pipette may be used to mix well. Five microliters of conjugate is pipetted onto the center of the conjugate pad. A single strip is dropped into each test tube for 15 minutes at room temperature. The flow of the sample and conjugate, the color of the membrane, and the formation of the test and control lines is observed. The positive sample should produce pink lines on the test and control reagents. The negative sample should only produce color on the control reagent. The sample flowing up the strip should visibly reach the wick, and the membrane background should be left white. The strip is removed from the tube, the wicking pad is peeled back, and the conjugate pad is removed. The results are visually evaluated using the DCN grading scale. Table I shows conditions used in lateral flow assays in this example.

TABLE I

| | | | | | Antigen | | | |
|---|---|---|---|---|---|---|---|---|
| Date | Strip no: | Membrane | Test Line Ab | Conjugate Ab | Standard | Serum | Concentration (ng/ml) | Sample Diluent |
| 14 Sep. 2011 | 1 | CN140 | IgG (0.7 mg/ml) | IgM | | | 0 | PBS+ |
| | 2 | CN140 | IgG (0.7 mg/ml) | IgM | X | | 10 | PBS+ |
| | 3 | CN140 | IgG (0.7 mg/ml) | IgM | X | | 100 | PBS+ |
| | 4 | CN140 | IgG (0.7 mg/ml) | IgM | X | | 1000 | PBS+ |
| | 5 | AE99 | IgG (0.7 mg/ml) | IgM | | | 0 | PBS+ |
| | 6 | AE99 | IgG (0.7 mg/ml) | IgM | X | | 10 | PBS+ |
| | 7 | AE99 | IgG (0.7 mg/ml) | IgM | X | | 100 | PBS+ |
| | 8 | AE99 | IgG (0.7 mg/ml) | IgM | X | | 1000 | PBS+ |
| | 9 | HF180 | IgG (0.7 mg/ml) | IgM | | | 0 | PBS+ |
| | 10 | HF180 | IgG (0.7 mg/ml) | IgM | X | | 10 | PBS+ |
| | 11 | HF180 | IgG (0.7 mg/ml) | IgM | X | | 100 | PBS+ |
| | 12 | HF180 | IgG (0.7 mg/ml) | IgM | X | | 1000 | PBS+ |
| | 13 | HF090 | IgG (0.7 mg/ml) | IgM | | | 0 | PBS+ |
| | 14 | HF090 | IgG (0.7 mg/ml) | IgM | X | | 10 | PBS+ |
| | 15 | HF090 | IgG (0.7 mg/ml) | IgM | X | | 100 | PI35+ |
| | 16 | HF090 | IgG (0.7 mg/ml) | IgM | X | | 1000 | PBS+ |
| 15 Sep. 2011 | 1 | CN140 | IgG (0.7 mg/ml) | IgM | | 0086 | Negative/Low positive | PBS+ |
| | 2 | CN140 | IgG (0.7 mg/ml) | IgM | | 0088 | Negative/Low positive | PBS+ |
| | 3 | CN140 | IgG (0.7 mg/ml) | IgM | | 5540-HP | High positive | PBS+ |
| | 4 | CN140 | IgG (0.7 mg/ml) | IgM | | 0354-MP | Medium positive | PBS+ |
| | 5 | CN140 | IgG (0.7 mg/ml) | IgM | X | | 1000 | PBS+ |
| | 6 | CN140 | IgG (0.7 mg/ml) | IgM | | | 0 | PBS+ |
| | 7 | CN140 | IgG (0.7 mg/ml) | IgM | | 5540-HP | | PBS+ |
| | 8 | CN140 | IgG (0.7 mg/ml) | IgM | | 0086 | | PBS+ |
| | 9 | CN140 | IgG (0.7 mg/ml) | IgM | | | 0 | PBS+ |
| | 10 | CN140 | IgG (0.7 mg/ml) | IgM | | 5540-HP | | HIV Running Buffer |
| | 11 | CN140 | IgG (0.7 mg/ml) | IgM | | 0086 | | HIV Running Buffer |
| | 12 | CN140 | IgG (0.7 mg/ml) | IgM | | | 0 | HIV Running Buffer |
| | 13 | CN140 | IgG (0.7 mg/ml) | IgM | | 5540-HP | | Running Buffer A |
| | 14 | CN140 | IgG (0.7 mg/ml) | IgM | | 0086 | | Running Buffer A |
| | 15 | CN140 | IgG (0.7 mg/ml) | IgM | | | 0 | Running Buffer A |
| | 16 | CN140 | IgG (0.7 mg/ml) | IgM | | 5540-HP | | Running Buffer B |
| | 17 | CN140 | IgG (0.7 mg/ml) | IgM | | 0086 | | Running buffer B |
| | 18 | CN140 | IgG (0.7 mg/ml) | IgM | | | 0 | Running buffer B |
| | 19 | CN140 | IgG (0.7 mg/ml) | IgM | | 5540-HP | | Running buffer C |
| | 20 | CN140 | IgG (0.7 mg/ml) | IgM | | 1186 | | Running buffer C |
| | 21 | CN140 | IgG (0.7 mg/ml) | IgM | | | 0 | Running Buffer C |
| | 22 | CN140 | IgG (1.4 mg/mL) | IgM | | 5540-HP | | HIV Running Buffer |
| | 23 | CN140 | IgG (1.4 mg/mL) | IgM | | 1186 | | HIV Running Buffer |
| | 24 | CN140 | IgG (1.4 mg/mL) | IgM | | | 0 | HIV Running Buffer |
| | 25 | CN140 | IgG (1.4 mg/mL) | IgM | | 5540-HP | | Running Buffer B |
| | 26 | CN140 | IgG (1.4 mg/mL) | IgM | | 1186 | | Running Buffer B |
| | 27 | CN140 | IgG (1.4 mg/mL) | IgM | | | 0 | Running Buffer B |
| | 28 | CN140 | IgM (0.5 mg/ml Spot) | IgG | | | 0 | PBS+ |
| | 29 | CN140 | IgM (0.5 mg/ml Spot) | IgG | X | | 10 | PBS+ |
| | 30 | CN140 | IgM (0.5 mg/ml Spot) | IgG | X | | 100 | PBS+ |
| | 31 | CN140 | IgM (0.5 mg/ml Spot) | IgG | X | | 1000 | PBS+ |

The anti-vitronectin IgG capture antibody on nitrocellulose membrane paired with an anti-vitronectin IgM detector antibody conjugated to colloidal gold works well in this lateral flow assay. The assay is functional with both Running buffer B and the HIV running buffer. The system consistently detects the target analyte in serum without background discoloration or nonspecific binding, and distinguishes between various concentrations.

Various modifications of the present invention, in addition to those shown and described herein, will be apparent to those skilled in the art of the above description. Such modifications are also intended to fall within the scope of the appended claims.

### Example 7

FIG. 6 is a graph showing age dependent differences in total vitronectin (VTN) concentrations in human serum samples. Quantitative VTN ELISA was performed on n=27 dengue fever (DF) and n=25 dengue hemorrhagic fever (DHF) from Puerto Rico Enhanced Surveillance Project located in Guayama. The samples were sub-divided by age and disease state (<15 years old and 15-19 years old; DF and DHF). The samples were diluted 1:50,000 and the concentrations of Vn were determined using the back calculation from the O.D. values using the standard curve. Normal healthy adult controls (>16 years of age but not age matched) were used in the assay. The results indicated that individuals <15 years of age do not have measurable differences in VTN between DF and DHF compared to patients 15-19 years of age. Results of patients 15-19 are also representative of results in adults older than 19 years of age.

Sequences

Human vitronectin precursor-NCBI Reference Sequence:
NP_000629.3-478 aa
SEQ ID NO: 1
MAPLRPLLILALLAWVALADQESCKGRCTEGFNVDKKCQCDELCSYYQSCCTDYTAECKPQVTRGDVF
TMPEDEYTVYDDGEEKNNATVHEQVGGPSLTSDLQAQSKGNPEQTPVLKPEEEAPAPEVGASKPEGID
SRPETLHPGRPOPPAFEELCSGKPFDAFTDLKNGSLFAFRGQYCYELDEKAVRPGYPKLIRDVWGIEG
PIDAAFTRINCQGKTYLFKGSQYWRFEDGVLDPDYPRNISDGFDGIPDNVDAALALPAHSYSGRERVY
FFKGKQYWEYQFQHQPSQEECEGSSLSAVFEHFAMMQRDSWEDIFELLFWGRTSAGTRQPQFISRDWH
GVPGQVDAAMAGRIYISGMAPRPSLAKKQRFRHRNRKGYRSQRGHSRGRNQNSRRPSRATWLSLFSSE
ESNLGANNYDDYRMDWLVPATCEPIQSVFFFSGDKYYRVNLRTRRVDTVDPPYPRSIAQYWLGCPAPG
HL
Nucleic acid sequence encoding human vitronectin precursor-1434
nucleotides
SEQ ID NO: 2
atggcacccctgagaccccttctcatactggccctgctggcatgggttgctctggctgaccaagagtc
atgcaagggccgctgcactgagggcttcaacgtggacaagaagtgccagtgtgacgagctctgctctt
actaccagagctgctgcacagactatacggctgagtgcaagccccaagtgactcgcggggatgtgttc
actatgccggaggatgagtacacggtctatgacgatggcgaggagaaaaacaatgccactgtccatga
acaggtggggggcccctccctgacctctgacctccaggcccagtccaaagggaatcctgagcagacac
ctgttctgaaacctgaggaagaggcccctgcgcctgaggtgggcgcctctaagcctgaggggatagac
tcaaggcctgagacccttcatccagggagacctcagcccccagcagaggaggagctgtgcagtgggaa
gcccttcgacgccttcaccgacctcaagaacggttccctctttgccttccgagggcagtactgctatg
aactggacgaaaaggcagtgaggcctgggtaccccaagctcatccgagatgtctggggcatcgagggc
cccatcgatgccgccttcacccgcatcaactgtcaggggaagacctacctcttcaagggtagtcagta
ctggcgctttgaggatggtgtcctggaccctgattacccccgaaatatctctgacggcttcgatggca
tcccggacaacgtggatgcagccttggccctccctgcccatagctacagtggccgggagcgggtctac
ttcttcaaggggaaacagtactgggagtaccagttccagcaccagcccagtcaggaggagtgtgaagg
cagctccctgtcggctgtgtttgaacactttgccatgatgcagcgggacagctgggaggacatcttcg
agcttctcttctggggcagaacctctgctggtaccagacagccccagttcattagccgggactggcac
ggtgtgccagggcaagtggacgcagccatggctggccgcatctacatctcaggcatggcaccccgccc
ctccttggccaagaaacaaaggtttaggcatcgcaaccgcaaaggctaccgttcacaacgaggccaca
gccgtggccgcaaccagaactcccgccggccatcccgcgccacgtggctgtccttgttctccagtgag
gagagcaacttgggagccaacaactatgatgactacaggatggactggcttgtgcctgccacctgtga
acccatccagagtgtcttcttcttctctggagacaagtactaccgagtcaatcttcgcacacggcgag
tggacactgtggaccctccctacccacgctccatcgctcagtactggctgggctgcccagctcctggc
catcgt
Mature human vitronectin-459 aa
SEQ ID NO: 3
DQESCKGRCTEGFNVDKKCQCDELCSYYQSCCTDYTAECKPQVTRGDVFTMPEDEYTVYDDGEEKNNA
TVHEQVGGPSLTSDLQAQSKGNPEQTPVLKPEEEAPAPEVGASKPEGIDSRPETLHPGRPQPPAEEEL
CSGKPFDAFTDLKNGSLFAFRGQYCYELDEKAVRPGYPKLIRDVWGIEGPIDAAFTRINCQGKTYLFK
GSQYWRFEDGVLDPDYPRNISDGFDGIPDNVDAALALPAHSYSGRERVYFFKGKQYWEYQFQHQPSQE
ECEGSSLSAVFEHFAMMQRDSWEDIFELLFWGRTSAGTRQPQFISRDWHGVPGQVDAAMAGRIYISGM
APRPSLAKKQRFRHRNRKGYRSQRGHSRGRNQNSRRPSRATWLSLFSSEESNLGANNYDDYRMDWLVP
ATCEPIQSVFFFSGDKYYRVNLRTRRVDTVDPPYPRSIAQYWLGCPAPGHL
Nucleic acid sequence encoding mature human vitronectin-1377
nucleotides
SEQ ID NO: 4
gaccaagagtcatgcaagggccgctgcactgagggcttcaacgtggacaagaagtgccagtgtgacga
gctctgctcttactaccagagctgctgcacagactatacggctgagtgcaagccccaagtgactcgcg
gggatgtgttcactatgccggaggatgagtacacggtctatgacgatggcgaggagaaaaacaatgcc
actgtccatgaacaggtggggggcccctccctgacctctgacctccaggcccagtccaaagggaatcc
tgagcagacacctgttctgaaacctgaggaagaggcccctgcgcctgaggtgggcgcctctaagcctg
aggggatagactcaaggcctgagacccttcatccagggagacctcagcccccagcagaggaggagctg
tgcagtgggaagcccttcgacgccttcaccgacctcaagaacggttccctctttgccttccgagggca
gtactgctatgaactggacgaaaaggcagtgaggcctgggtaccccaagctcatccgagatgtctggg
gcatcgagggccccatcgatgccgccttcacccgcatcaactgtcaggggaagacctacctcttcaag
ggtagtcagtactggcgctttgaggatggtgtcctggaccctgattacccccgaaatatctctgacgg
cttcgatggcatcccggacaacgtggatgcagccttggccctccctgcccatagctacagtggccggg
agcgggtctacttcttcaaggggaaacagtactgggagtaccagttccagcaccagcccagtcaggag
gagtgtgaaggcagctccctgtcggctgtgtttgaacactttgccatgatgcagcgggacagctggga
ggacatcttcgagcttctcttctggggcagaacctctgctggtaccagacagccccagttcattagcc
gggactggcacggtgtgccagggcaagtggacgcagccatggctggccgcatctacatctcaggcatg
gcaccccgcccctccttggccaagaaacaaaggtttaggcatcgcaaccgcaaaggctaccgttcaca
acgaggccacagccgtggccgcaaccagaactcccgccggccatcccgcgccacgtggctgtccttgt
tctccagtgaggagagcaacttgggagccaacaactatgatgactacaggatggactggcttgtgcct
gccacctgtgaacccatccagagtgtcttcttcttctctggagacaagtactaccgagtcaatcttcg
cacacggcgagtggacactgtggaccctccctacccacgctccatcgctcagtactggctgggctgcc
cagctcctggccatctg

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process for assessing dengue virus infection in a human subject comprising:
obtaining a biological sample from the human subject; and
quantifying vitronectin in the sample, wherein the amount of vitronectin present in the sample is indicative of the severity of dengue virus infection in the human subject.

2. The process of claim 1, wherein quantifying vitronectin comprises immunoassay and/or mass spectrometry.

3. The process of claim 1, wherein the biological sample is selected from the group consisting of: whole blood, plasma, serum, extracellular fluid, cytosolic fluid, and tissue.

4. The process of claim 1, further comprising purifying vitronectin from the biological sample prior to quantifying vitronectin.

5. The process of claim 2, wherein the immunoassay is an ELISA or an antigen capture assay.

6. The process of claim 5, wherein the antigen capture assay is a lateral flow assay.

7. A process for assessing dengue virus infection in a human subject comprising:
obtaining a first biological sample from the human subject at a first time during the acute febrile phase of dengue virus infection;
obtaining a second biological sample from the human subject at a second time later than the first time during the acute febrile phase or critical phase of dengue virus infection;
quantifying vitronectin in the first biological sample to obtain a first vitronectin level;
quantifying vitronectin in the second biological sample to obtain a second vitronectin level; and
comparing the first vitronectin level and the second vitronectin level to assess dengue virus infection in the human subject, wherein a decrease in the second vitronectin level compared to the first vitronectin level indicates that dengue virus infection is progressing from dengue fever to dengue hemorrhagic fever.

8. The process of claim 7, wherein quantifying vitronectin comprises immunoassay and/or mass spectrometry.

9. The process of claim 7, wherein the first biological sample and the second biological sample are selected from the group consisting of: whole blood, plasma, serum, extracellular fluid, cytosolic fluid, and tissue.

10. The process of claim 7, further comprising purifying vitronectin from the first biological sample and the second biological sample prior to quantifying vitronectin.

11. The process of claim 8, wherein the immunoassay is an ELISA or an antigen capture assay.

12. The process of claim 11, wherein the antigen capture assay is a lateral flow assay.

13. A vitronectin immunoassay device, comprising:
a solid or semi-solid porous support comprising a binding agent capable of specific binding to a first epitope of vitronectin.

14. The vitronectin immunoassay device of claim 13, further comprising a conjugate pad comprising a detectably labeled binding agent capable of specific binding to a second epitope of vitronectin.

15. The vitronectin immunoassay device of claim 13, further comprising a conjugate pad comprising a detectably labeled vitronectin.

16. The vitronectin immunoassay device of claim 14,
further comprising a wicking pad.

17. The vitronectin immunoassay device of claim 13, further comprising a housing at least partially enclosing the conjugate pad, the solid or semi-solid porous support, and/or the wicking pad.

18. A process for assessing a febrile illness in a human subject comprising:
obtaining a serum, plasma or whole blood sample from the human subject;
quantitating vitronectin in the sample to determine the level of vitronectin in the sample;
and comparing the level of vitronectin with a standard or control to differentiate dengue fever or other febrile illness from severe dengue.

19.-20. (canceled)
